SEQUENCE LISTING (10 sequences)

SEQ ID NO: 1 - RNA, 23 nt, synthetic construct
  source 1..23: mol_type = other RNA
ttctccgaacgtgtcacgtdtdt

SEQ ID NO: 2 - RNA, 23 nt, synthetic construct
  source 1..23: mol_type = other RNA
acgtgacacgttcggagaadtdt

SEQ ID NO: 3 - RNA, 23 nt, synthetic construct
  source 1..23: mol_type = other RNA
ccaatagtctaacttaaaadtdt

SEQ ID NO: 4 - RNA, 23 nt, synthetic construct
  source 1..23: mol_type = other RNA
ttttaagttagactattggdtdt

SEQ ID NO: 5 - RNA, 23 nt, synthetic construct
  source 1..23: mol_type = other RNA
gagatgaaaagcacatctadtdt

SEQ ID NO: 6 - RNA, 23 nt, synthetic construct
  source 1..23: mol_type = other RNA
tagatgtgcttttcatctcdtdt

SEQ ID NO: 7 - RNA, 23 nt, synthetic construct
  source 1..23: mol_type = other RNA
ctctcccaaatatttgaaadtdt

SEQ ID NO: 8 - RNA, 23 nt, synthetic construct
  source 1..23: mol_type = other RNA
tttcaaatatttgggagagdtdt

SEQ ID NO: 9 - RNA, 23 nt, synthetic construct
  source 1..23: mol_type = other RNA
ggagtttcaaccaaagatadtdt

SEQ ID NO: 10 - RNA, 23 nt, synthetic construct
  source 1..23: mol_type = other RNA
tatctttggttgaaactccdtdt

(19) United States
(12) Patent Application Publication
(10) Pub. No.: US 2026/0335245 A1
(43) Pub. Date: Oct. 29, 2026

(54) MACROPHAGE-TARGETED SMALL ACTIVATING RIBONUCLEIC ACID NANOMEDICINE FOR INFLAMMATORY MICROENVIRONMENT, PREPARATION METHOD AND APPLICATION THEREOF

(71) Applicant: Hengqin Hospital of the First Affiliated Hospital of Guangzhou Medical University, Zhuhai (CN)

(72) Inventors: Lingmin Zhang, Guangzhou (CN); Yingjie Gong, Guangzhou (CN); Wenhua Liang, Zhuhai (CN); Aiping Qin, Guangzhou (CN); Shitao Lin, Zhuhai (CN)

(21) Appl. No.: 19/537,983

(22) Filed: Feb. 12, 2026

(30) Foreign Application Priority Data
Apr. 18, 2025 (CN) 202510491050.2

(51) Int. Cl. A61K 9/48; A61K 9/00; A61K 31/7105; A61K 48/00; A61P 29/00

(52) U.S. Cl. CPC A61K 9/4833; A61K 9/0019; A61K 31/7105; A61K 48/0041; A61P 29/00

(57) ABSTRACT

A macrophage-targeted small activating ribonucleic acid (saRNA) nanomedicine for inflammatory microenvironment includes a saRNA core and a liposome shell. After the macrophage-targeted saRNA nanomedicine for inflammatory microenvironment is actively targeted to inflammatory microenvironment and internalized into cells via receptor recognition, the liposome shell is swollen and disintegrated, releasing saRNAs. The saRNAs are utilized to regulate gene expression, reprogram M1 macrophages into M2 macrophages in terms of phenotype and metabolic pattern, and promote the secretion of anti-inflammatory factors, thereby achieving the effect of inflammation inhibition. Therefore, the present disclosure has broad application prospects in gene therapy and immunotherapy for inflammatory and infectious diseases.

### CROSS REFERENCE TO RELATED APPLICATION

This patent application claims the benefit and priority of Chinese Patent Application No. 202510491050.2 filed with the China National Intellectual Property Administration on Apr. 18, 2025, the disclosure of which is incorporated by reference herein in its entirety as part of the present application.

### REFERENCE TO SEQUENCE LISTING

A computer readable XML file entitled “HKIP-US-1-1312-65-Sequence Listing”, that was created on Jan. 23, 2026, with a file size of about 10,203 bytes, contains the sequence listing for this application, has been filed with this application, and is hereby incorporated by reference in its entirety.

### TECHNICAL FIELD

The present disclosure relates to the technical field of biomedicine, and in particular to a macrophage-targeted small activating ribonucleic acid (saRNA) nanomedicine for inflammatory microenvironment, a preparation method and an application thereof.

### BACKGROUND

Pneumonia refers to the inflammation of the terminal airway, alveoli and lung interstitium, and is a landmark pathological feature of lung injury caused by exogenous or endogenous blows to the lungs. As the main cellular component of the inflammatory response, macrophages gather in large numbers in the inflammatory microenvironment. After being activated by inflammatory mediators, macrophages attack normal cells in the inflammatory area. As an innate immune cell, the polarization process of macrophages plays an important role in affecting the lung environment. Macrophages activated by different cytokines and chemokines can be divided into M1 pro-inflammatory macrophages and M2 anti-inflammatory macrophages, which can further produce different effects: M1 macrophages can be induced by interferon γ (IFN-γ), lipopolysaccharide (LPS) and tumor necrosis factor-α (TNFα), and have the ability to enhance antigen presentation, produce nitric oxide and secrete a large number of pro-inflammatory factors; and M2 macrophages can be induced by colony stimulating factor 1 (CSF-1), interleukin-1β (IL1β), interleukin-4 (IL4) and interleukin-10 (IL10), which can inhibit the secretion of pro-inflammatory factors in M1 macrophages, promote the healing of injured lung tissue, and regulate the functional balance of macrophages. Studies have shown that regulating the M1/M2 balance of macrophages and reprogramming macrophages is an important means to treat pulmonary inflammatory diseases.

Gene therapy refers to a new therapeutic method of treating diseases by introducing external genes at the gene level to realize changes such as substitution, replacement, knock-in/knock-out, activation/inhibition, and the like of genes in vivo. SaRNA is a new type of gene therapy drug, which can stimulate gene expression at the transcription level, and has the characteristics of small molecular weight, gene specificity and activation of gene expression. Studies have shown that upregulating peroxisome proliferator-activated receptor γ (PPARγ) can reprogram M1 macrophages into M2 macrophages.

Like most nucleic acid drugs, and similar to other RNA drugs, saRNA still has limitations in specificity, stability and delivery means. Although chemical modification can improve its stability and specificity, it is accompanied by material toxicity. Therefore, further in-depth research on the delivery system of saRNA is required. Liposomes show great advantages in systemic or local delivery of RNA: due to their low toxicity and immunogenicity, high biocompatibility and biodegradability of components such as phospholipids, liposomes can increase the concentration of drugs in vivo while protecting drugs from degradation; and secondly, liposomes are easily modified with various ligands and functional molecules, which can greatly improve the targeting of RNA. How to reduce the non-specific clearance of nucleic acid drug carriers and improve the tissue/cell targeting of nucleic acid drug carriers is a hot and difficult point in nucleic acid drug delivery.

### SUMMARY

The present disclosure provides a preparation method of a macrophage-targeted saRNA nanomedicine for inflammatory microenvironment, including the steps of:
(1) dissolving SM-102, 1,2-distearoyl-sn-glycero-3-phosphocholine (DSPC), cholesterol, 1,2-dimyristoyl-rac-glycero-3-methoxypolyethylene glycol-2000 (DMG-PEG2000) and TKPR-polyethylene glycol 2000-distearoylphosphatidylethanolamine (TKPR-PEG2000-DSPE) in an organic solvent at a molar ratio of 45-55:8-12:30-42:1-2:1-2; removing the organic solvent by rotary evaporation; and adding 1-3 mL of buffer solution for hydration to adjust a total concentration of SM-102, DSPC, Cholesterol, DMG-PEG2000 and TKPR-PEG2000-DSPE in a mixed solution to 0.4-0.6 mg/mL, and forming a liposome solution; and
(2) diluting 0.8-1.2 μg saRNA in 0.8-1.2 μL of ribonuclease (RNase)-free water, and complexing the diluted saRNA with the liposome solution at a mass ratio of liposome solution to saRNA of 38-42:0.8-1.2, followed by standing at room temperature and centrifuging to obtain a precipitate, which is a macrophage-targeted saRNA nanomedicine for inflammatory microenvironment.

Sequences of sense and antisense strands of the saRNA are shown in SEQ ID NOs.3 and 4.

In some examples of the present disclosure, a molar ratio of SM-102, DSPC, cholesterol, DMG-PEG2000, and TKPR-PEG2000-DSPE in step (1) is 50:10:37:1.5:1.5.

In some examples of the present disclosure, the organic solvent in step (1) is a mixed liquid of chloroform and methanol.

In some examples of the present disclosure, a volume ratio of the chloroform and methanol in step (1) is 3:1.

In some examples of the present disclosure, the buffer solution in step (1) is 8-12 nM citrate buffer solution.

In some examples of the present disclosure, a total concentration of SM-102, DSPC, cholesterol, DMG-PEG2000, and TKPR-PEG2000-DSPE in the mixed solution in step (1) is 0.5 mg/mL.

In some examples of the present disclosure, in step (2), 1 μg of saRNA is diluted in 1 μL of RNase-free water.

In some examples of the present disclosure, in step (2), a mass ratio of liposome solution to saRNA is 40:1.

The present disclosure also provides a macrophage-targeted saRNA nanomedicine for inflammatory microenvironment prepared by the preparation method.

The present disclosure also provides an application of the macrophage-targeted saRNA nanomedicine for inflammatory microenvironment in preparing anti-pneumonia drugs.

Compared with the related art, the present disclosure has the following beneficial effects.

In the present disclosure, the macrophage-targeted saRNA nanomedicine for inflammatory microenvironment is prepared by loading saRNA for PPARγ (sa-PPARγ) into liposomes modified with the M1 macrophage-targeting peptide TKPR. The nanomedicine recognizes neuropilin-1 (Nrp-1) receptor into M1 macrophages under the mediation of TKPR-targeting peptide, releases saPPARγ, and significantly up-regulates the expression of PPARγ. The phenotype and metabolic mode of M1 macrophages are reprogrammed into M2 macrophages, thereby releasing anti-inflammatory factors and down-regulating the level of pro-inflammatory factors, and finally achieving the objective of reducing inflammation. It can be seen that the present disclosure provides a new drug for the treatment of pneumonia.

### BRIEF DESCRIPTION OF THE DRAWINGS

FIG. 1A shows expression levels of PPARγ messenger RNA (mRNA) in macrophages transfected with different sequences of saRNA, detected by quantitative real-time polymerase chain reaction (qRT-PCR).

FIG. 1B shows expression levels of PPARγ protein in macrophages transfected with different sequences of saRNA, detected by Western Blot (WB).

FIG. 2A shows the cell morphology of M1 macrophages and those transfected with saPPARγ observed by optical microscopy, with a scale bar of 200 μm.

FIG. 2B shows protein expression levels in M1 macrophages transfected with saPPARγ detected by WB.

FIG. 2C shows mRNA expression levels in M1 macrophages transfected with saPPARγ detected by qRT-PCR.

FIG. 2D shows mRNA expression levels in M1 macrophages transfected with different concentrations of saPPARγ detected by qRT-PCR.

FIG. 3A shows comparison of particle size and zeta potential of targeted liposomal particles (TLPs) at different mass ratios.

FIG. 3B shows gel retardation of TLPs at different mass ratios.

FIG. 3C shows nucleic acid protection effect of TLPs at different mass ratios.

FIG. 3D shows encapsulation efficiency of TLPs at different mass ratios.

FIG. 3E shows cellular uptake of liposomes with different electrical properties, with a scale bar of 50 μm.

FIG. 3F shows particle size distribution and morphology of TLPs at a mass ratio=40.

FIG. 3G shows three-day stability of TLPs in water at a mass ratio=40.

FIG. 3H shows drug release curves of TLPs under different pH conditions.

FIG. 4A shows cellular uptake of different formulations detected by laser scanning confocal microscopy (LSCM) and flow cytometry (FCM), with a scale bar of 50 μm.

FIG. 4B shows uptake of TLPs in different cells detected by LSCM and FCM, with a scale bar of 50 μm.

FIG. 4C shows lysosomal escape of TLPs observed by LSCM, with a scale bar of 50 μm.

FIG. 4D shows cell viability of macrophages transfected with different formulations observed by LSCM, with a scale bar of 200 μm.

FIG. 4E shows the effect of different formulations on macrophage viability detected by cell counting kit-8 (CCK-8) assay.

FIG. 4F shows the effect of different concentrations of saPPARγ on macrophage viability detected by CCK8 assay.

FIG. 4G shows protein expression levels in macrophages transfected with different formulations detected by WB.

FIG. 4H shows mRNA expression levels in macrophages transfected with different formulations detected by qRT-PCR.

FIG. 5A shows distribution and metabolism of different formulations in mice with pneumonia observed by In Vivo Small Animal Imaging.

FIG. 5B shows distribution of different formulations in major organs of mice with pneumonia observed by In Vivo Small Animal Imaging.

FIG. 5C shows comparison of fluorescence signals of different formulations in major organs of mice with pneumonia.

FIG. 6A shows results of lung histological analysis of a pneumonia treatment experiment with TLPs in Example 1.

FIG. 6B shows results of WB analysis of cluster of differentiation 80 (CD80), PPARγ, Arginase 1 (Arg1), and cluster of differentiation 206 (CD206) in the pneumonia treatment experiment with TLPs in Example 1.

FIG. 6C shows results of lung function testing in the pneumonia treatment experiment with TLPs in Example 1, specifically a tidal inspiratory time (TI) index.

FIG. 6D shows results of lung function testing in the pneumonia treatment experiment with TLPs in Example 1, specifically a minute volume (MV) index.

FIG. 7 is a schematic diagram of a technical route and a mechanism of TLPs of the present disclosure.

### DETAILED DESCRIPTION

### Example 1

### I. Method

### 1. Nucleic Acid Sequence Screening

M1 macrophages were cultured overnight and transfected with different sequences of saRNA using Polyethylenimine (PEI). The cells were further cultured for 48 hours, and total cellular RNA was extracted. The expression level of PPARγ was analyzed by qRT-PCR. The saRNA sequence is as follows:

saRNANC:
sense:
(SEQ ID NO. 1)
5′-UUCUCCGAACGUGUCACGUdTdT-3′
anti-sense:
(SEQ ID NO. 2)
5′-ACGUGACACGUUCGGAGAAdTdT-3′
sa-PPARγ-1:
sense:
(SEQ ID NO. 3)
5′-CCAAUAGUCUAACUUAAAAdTdT-3′
anti-sense:
(SEQ ID NO. 4)
5′-UUUUAAGUUAGACUAUUGGdTdT-3′
sa-PPARγ-2:
sense:
(SEQ ID NO. 5)
5′-GAGAUGAAAAGCACAUCUAdTdT-3′
anti-sense:
(SEQ ID NO. 6)
5′-UAGAUGUGCUUUUCAUCUCdTdT-3′
sa-PPARγ-3:
sense:
(SEQ ID NO. 7)
5′-CUCUCCCAAAUAUUUGAAAdTdT-3′
anti-sense:
(SEQ ID NO. 8)
5′-UUUCAAAUAUUUGGGAGAGdTdT-3′
sa-PPARγ-4:
sense:
(SEQ ID NO. 9)
5′-GGAGUUUCAACCAAAAGAUAdTdT-3′
anti-sense:
(SEQ ID NO. 10)
5′-UAUCUUUGGUUGAAACUCCdTdT-3′

### 2. Regulation of Macrophage Polarization by PPARγ

### 2.1. Morphological Changes of Macrophages

M1 macrophages were cultured overnight and transfected with saPPARγ using PEI. The cells were further cultured for 48 hours, and the cell morphology was observed under an optical microscope.

### 2.2. Regulation of Macrophage Polarization by PPARγ

M1 macrophages were cultured overnight and transfected with saPPARγ using PEI. The cells were further cultured for 48 hours, total cellular protein was extracted, and the expression levels of Arg1 and CD206 were analyzed by WB. Total cellular RNA was also extracted, and the expression levels of Arg1 and CD206 were analyzed by qRT-PCR.

M1 macrophages were cultured overnight and transfected with different concentrations of saPPARγ using PEI. The cells were further cultured for 48 hours, total cellular RNA was extracted, and the expression levels of PPARγ, CD206, and IL-10 (IL-10) were analyzed by qRT-PCR.

### 3. Synthesis and Characterization of TLPs

### 3.1. Synthesis of Targeting Liposomes (TLs)

Thin film hydration method was adopted: 5 components including SM-102, DSPC, cholesterol, DMG-PEG2000, and TKPR-PEG2000-DSPE, were used to synthesize liposomes. The components were weighed according to a molar ratio of 50/10/37/1.5/1.5 and dissolved in a mixed solution of chloroform/methanol (3/1, v/v). The organic solvent was removed by rotary evaporation on a rotary evaporator for 30 minutes. Subsequently, 2 mL of 10 nM citrate buffer (pH=3) was added for hydration, and a total concentration of SM-102, DSPC, cholesterol, DMG-PEG2000, and TKPR-PEG2000-DSPE in the mixed solution reached 0.5 mg/mL. The mixture was extruded back and forth using an extruder to form a TLs solution, which was stored at 4° C. for later use.

Targeting liposome-saRNANC complex (TLC) and liposome-saRNA particles without targeting peptide (LPs) were used as controls. TLC was formed by complexing TLs with saRNANC at a mass ratio (TL:saRNA) of 40:1. A liposome solution was prepared using SM-102, DSPC, cholesterol, and DMG-PEG2000 at a molar ratio of 50/10/37/3. This liposome solution was complexed with saRNA at a mass ratio (liposome solution:saRNA) of 40:1 to prepare nanoparticle particles without macrophage-targeting peptide, which was abbreviated as LP.

### 3.2. Screening of TLP Mass Ratios

1 μg of saRNA was diluted in 1 μL of RNase-free water to adjust a final concentration to 1 μg/L. The saRNA solution was complexed with the TL solution at mass ratios (TL:saRNA) of 10:1, 20:1, 30:1, 40:1, 50:1, 60:1, and 70:1. After standing at room temperature for 15 minutes, the mixture was centrifuged at 13,000 rpm for 30 minutes to remove unencapsulated saRNA. The supernatant was discarded after centrifugation, and 1 mL of phosphate-buffered saline (PBS) was added to resuspend the precipitate, which was stored at 4° C. for later use. Subsequently, 1 mL of double-distilled water was added to dilute the complex solution, and the particle size and zeta potential of the complex were measured using a Malvern particle size analyzer.

1 μg of saRNA was diluted in 1 μL of RNase-free water. The diluted saRNA was complexed with the TL solution at mass ratios (TL:saRNA) of 10:1, 20:1, 30:1, 40:1, 50:1, 60:1, and 70:1.

After standing at room temperature for 15 minutes, the mixture was centrifuged at 13,000 rpm for 30 minutes. 20 μL of the supernatant was collected, mixed with 10× Loading buffer, and loaded onto a gel. Electrophoresis was performed on a 1% agarose gel at 120 V for 20 minutes, and images were captured and saved using a gel imager. The encapsulation efficiency was calculated based on the results.

### 3.3. Drug Release

TLP was prepared using fluorescently labeled saRNA and loaded into a dialysis bag with a molecular weight cutoff (MWCO) of 3500. The dialysis bags were placed in PBS buffer solutions at pH 5.4 and pH 7.4. Dialysate samples were collected at 1, 2, 3, 4, 5, 6, 8, 10, 12, 24, 48, and 72 hours. The fluorescence intensity was measured using a fluorescence spectrophotometer, and the cumulative drug release rate was calculated.

### 4. TLPs-Mediated Macrophage Reprogramming

### 4.1. Cellular Uptake

TLPs were prepared with fluorescent dye-labeled saRNA. M1 macrophages were cultured overnight, and culture media including PBS, saRNA, LP, and TLPs were added. After incubation for 9 hours, the cellular uptake of different formulations was detected by FCM and LSCM.

### 4.2. Cell-Specific Uptake

Mouse alveolar epithelial cells (MLE), mouse embryonic fibroblasts (3T3), M0 macrophages, and M1 macrophages were cultured overnight. These cells were incubated with TLPs including fluorescently labeled saRNA of equal mass (60 nM) for 9 hours. The uptake of TLPs by different cell types was detected using FCM and LSCM.

### 4.3. Lysosomal Escape

M1 macrophages were cultured overnight and incubated with Targeted TLPs including fluorescently labeled saRNA of equal mass (60 nM) for 3 hours and 9 hours. The cells were gently washed twice with PBS and incubated with a green lysosomal probe. The lysosomal escape of the nanoparticles within the cells was observed using LSCM.

### 4.4. Cytotoxicity

M1 macrophages were cultured overnight and incubated with culture media including PBS, TLs, saRNA, TLC, LPs, and TLPs (the latter four groups each included 60 nM saRNA) for 8 hours. The culture medium was replaced, and the cells were further cultured for 48 hours. The cells were stained using a cell viability/cytotoxicity assay kit, and the cell survival and death status were observed under a LSCM to analyze the cytotoxicity of different formulations.

### 4.5. Macrophage Reprogramming Efficiency

M1 macrophages were cultured overnight and incubated with culture media including PBS, TLs, saRNA, TLC, LPs, and TLPs (the latter four groups each included 60 nM saRNA) for 8 hours. The culture medium was replaced, and the cells were further cultured for 48 hours. Total cellular protein was extracted, and the expression levels of inducible Nitric Oxide Synthase (iNOS), CD80, CD206, and PPARγ were analyzed by WB to evaluate the efficiency of TLPs in reprogramming macrophages.

### 5. In Vivo Anti-Inflammatory Efficacy of TLPs

### 5.1. Establishment of a Mouse Pneumonia Model

A mouse pneumonia model was established by intratracheal instillation of LPS (2 mg/kg) in C57BL/6J mice.

5.2. In vivo distribution

TLPs were prepared with Cy7-labeled saRNA (Cy7-saRNA). Four hours after intratracheal instillation of LPS (2 mg/kg), the mice were randomly divided into 4 groups and received orbital injection of PBS, saRNA, LPs, and TLPs (the latter three groups each included 20 μg Cy7-saRNA per 100 μL), and the injection dose for each mouse in all groups was 100 μL. In vivo imaging of the mice was performed using a small animal imaging system at 2, 6, 12, 24, and 48 hours to observe the in vivo distribution and metabolism of TLPs. After 48 hours, the mice were euthanized and dissected to collect the heart, liver, spleen, lungs, and kidneys, which were imaged to observe the distribution of TLPs in various organs.

### 5.3. Pneumonia Treatment Efficacy

The mice were randomly divided into 6 groups. Four hours after intratracheal instillation of LPS (2 mg/kg), except for the normal control group, the other 5 groups received orbital injection of normal saline, TLs, saRNA, LPs, and TLPs (the latter three groups each included 20 μg saPPARγ per 100 L), and the injection dose for each mouse in all groups was 100 μL. Administration was performed once every 3 days, totaling two doses. Three days after the last administration, lung function parameters of the mice were detected to evaluate the efficacy of TLPs in restoring lung function. The mice were euthanized and dissected. Lung tissues were collected for hematoxylin and eosin (H&E) staining to assess the therapeutic effect of TLPs. Total protein was extracted from the lung tissues, and the expression levels of Arg1, CD80, CD206, and PPARγ were analyzed by WB to evaluate the in vivo reprogramming efficiency of TLPs.

### II. Results

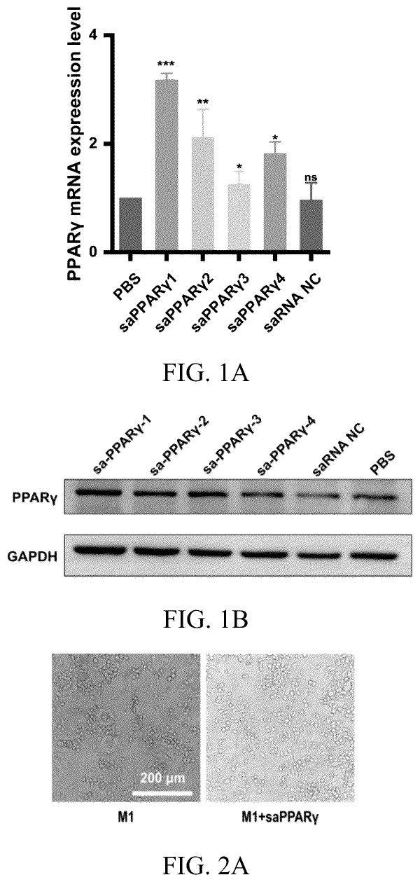

After M1 macrophages were transfected with different sequences of saRNA, the results of qRT-PCR showed that the expression of PPARγ in the cells transfected with sa-PPARγ-1 was most significantly upregulated (FIG. 1A), and the results of WB were consistent with this (FIG. 1). This indicated that sa-PPARγ-1 could effectively reprogram M1 macrophages, and this sequence was used in all subsequent experiments, which is as follows:

sense:
(SEQ ID NO. 3)
5′-CCAAUAGUCUAACUUAAAAdTdT-3′
anti-sense:
(SEQ ID NO. 4)
5′-UUUUAAGUUAGACUAUUGGdTdT-3′

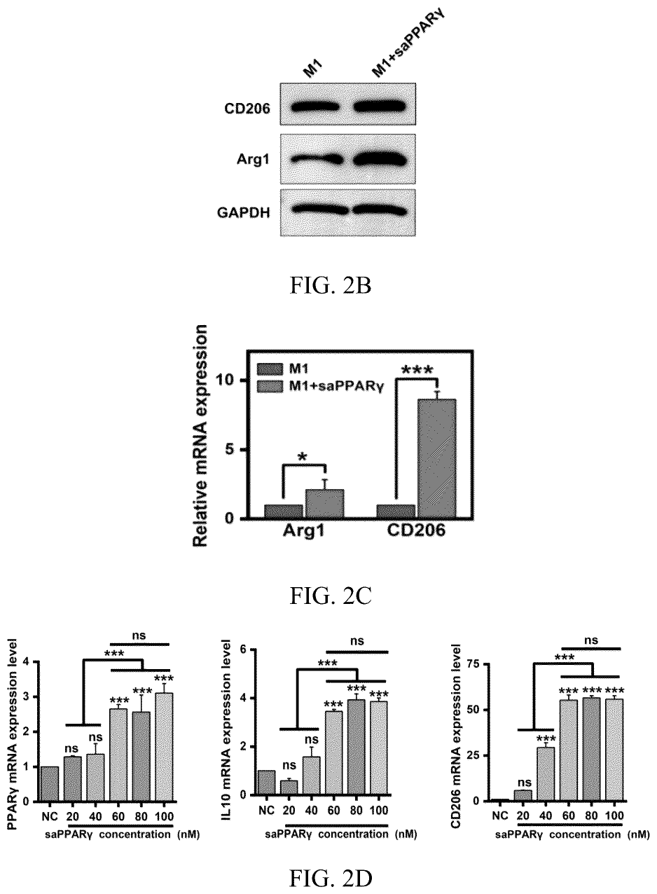

The morphology of macrophages before and after transfection was observed using an optical microscope. After transfection with saPPARγ, M1 macrophages were transformed into spindle-shaped M2 macrophages (FIG. 2A). After M1-type macrophages were transfected with saPPARγ, the protein and mRNA levels of the M2 macrophage markers CD206 and Arg1 were upregulated (FIGS. 2B and 2C). This indicated that PPARγ could regulate the polarization process of macrophages and polarize M1 macrophages into M2 macrophages. M1 macrophages were transfected with saPPARγ at different concentrations. It was found that when the concentration of saPPARγ reached 60 nM, the mRNA levels of PPARγ and the M2 macrophage markers CD206 and IL-10 were upregulated to the peak, and the expression levels did not change with the further increase in saPPARγ concentration thereafter. This indicated that saPPARγ at a concentration of 60 nM could effectively regulate the polarization of M1-type macrophages (FIG. 2D). Therefore, saPPARγ at 60 nM was used for TLP synthesis in subsequent experiments.

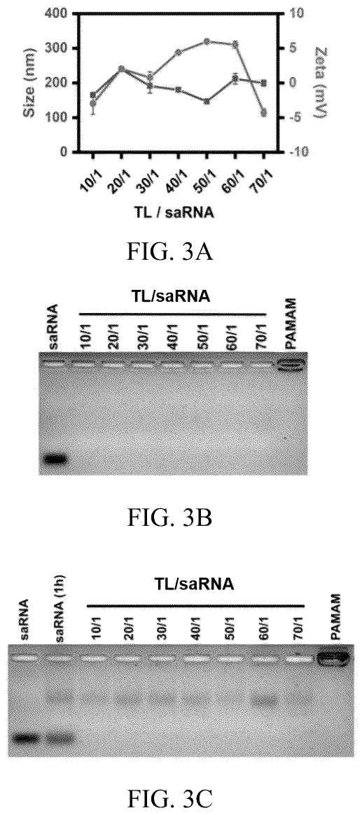

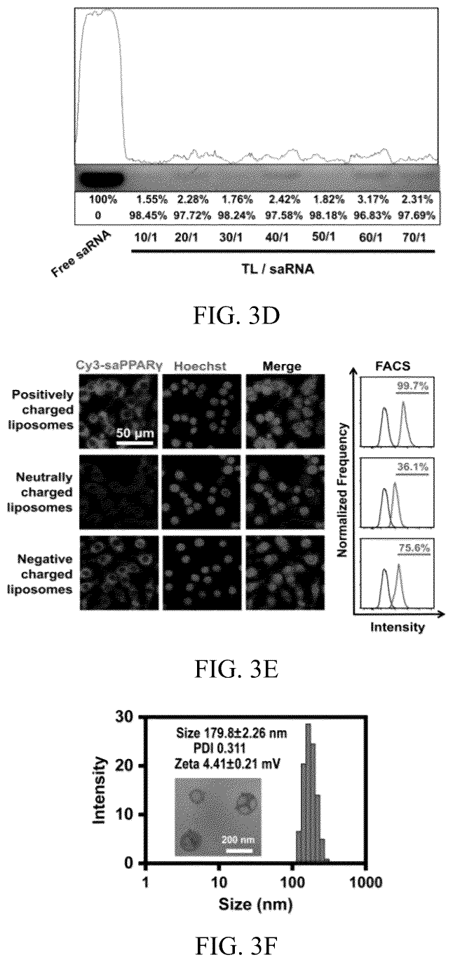

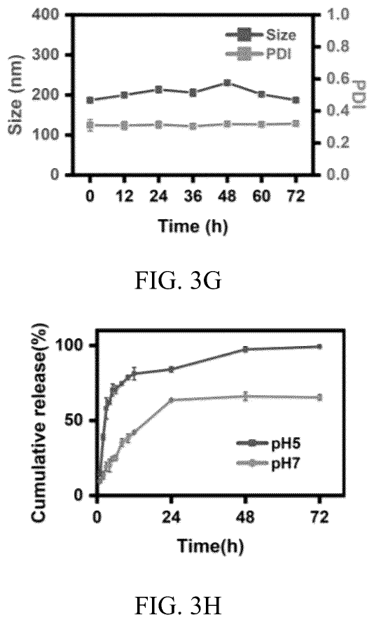

With the increase in mass ratio, the particle size of the liposome delivery system (TLP) reached a relatively small value at a mass ratio of 40, and its zeta potential was approximately 4 mV (FIG. 3A). In addition, its encapsulation efficiency was 97.58%, demonstrating that saRNA could be effectively loaded (FIG. 3D). TLPs with different mass ratios could effectively encapsulate saRNA within the liposomes (FIG. 3B) and still effectively protect saRNA after co-incubation with serum for 60 minutes (FIG. 3C). The uptake of liposomes with different electrical properties by M1 macrophages was observed using LSCM. The results showed that the uptake amount by M1 macrophages was relatively the highest when TLPs were positively charged (FIG. 3E). A mass ratio of 40 was selected for TLP preparation based on a comprehensive evaluation of particle size, zeta potential and cellular uptake performance. TLPs were round nanoparticles with distinct liposome morphology. Several cavities formed by SM-102 complexed with saRNA were observed in the liposome lumen under electron microscopy, which further confirmed the successful preparation of TLPs (FIG. 3F). Subsequently, the stability of TLPs in water for three days was investigated. The results showed that no significant changes were observed in its particle size and polydispersity index (PDI), indicating that TLPs had a relatively stable structure (FIG. 3G). The cumulative drug release rate of TLPS reached 99% within 72 hours in a pH 5.4 environment, while the cumulative release rate was approximately 60% in a pH 7.5 environment (FIG. 3H).

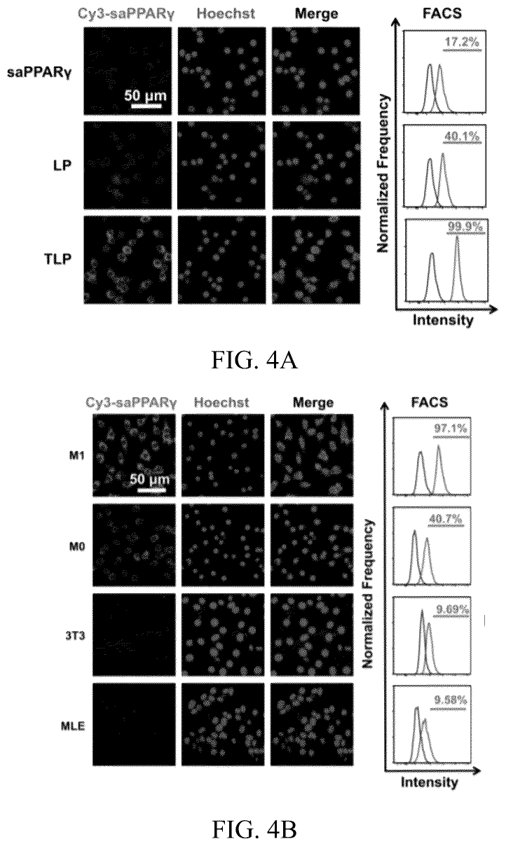

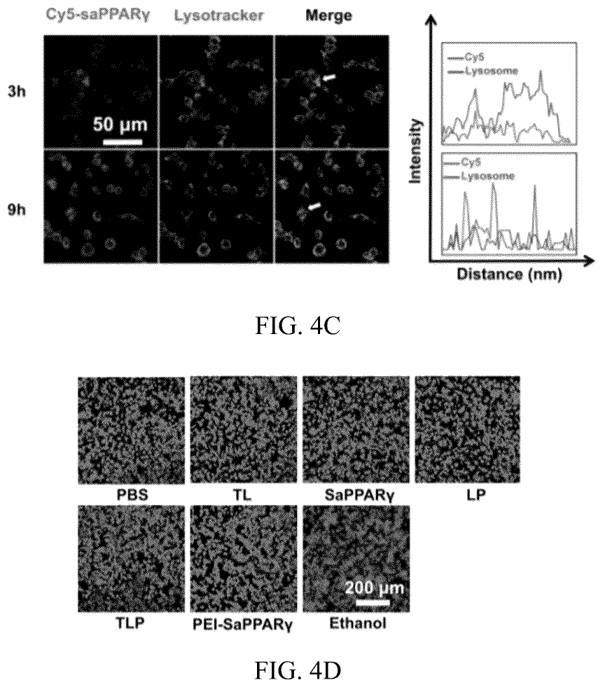

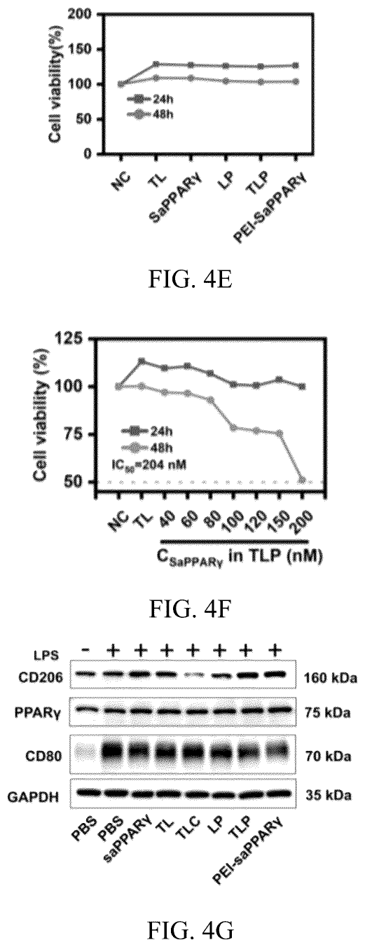

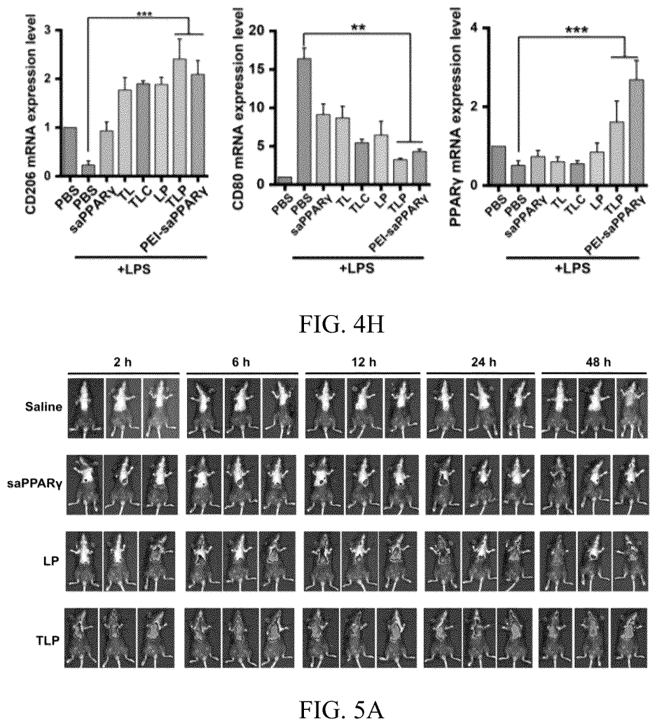

M1 macrophages were transfected with pure saRNA, LPs without targeting peptide, and TLPs. Compared with the other two groups, TLPs exhibited a stronger fluorescence signal inside the cells (FIG. 4A). TLPs were co-cultured overnight with MLE, 3T3, M0 macrophages, and M1 macrophages. The fluorescence intensity of TLPs in the first three types of cells was weaker than that in M1 macrophages, indicating that TLPs could effectively recognize M1 macrophages and possessed favorable M1 macrophage targeting ability (FIG. 4B). After 3 hours of co-incubation with cells, the fluorescence signal of TLPs overlapped with the green fluorescence signal of lysosomes, suggesting that TLPs were captured by lysosomes at this time. However, after 9 hours, the fluorescence signal of TLPs was separated from that of lysosomes, and the lysosome fluorescence signal was attenuated, demonstrating that TLPs had escaped from the lysosomes by then (FIG. 4C). 24 and 48 hours after M1 macrophages were transfected with TLPs, the cells still exhibited calcein-positive signals (FIG. 4D), and maintained good viability after transfection (FIG. 4E). This indicated that TLPs possessed low cytotoxicity. Meanwhile, CCK8 assay results showed that TLPs including different concentrations of saPPARγ exerted no significant effect on cell viability within 24 hours, and TLPs with high concentrations of saPPARγ exhibited an inhibitory effect on cell viability after 48 hours (FIG. 4F). After M1 macrophages were transfected with TLPs, the expression of PPARγ was upregulated, the level of CD206 (the marker protein of M2-type macrophages) was increased, and the level of iNOS (the marker of M1 macrophages) was decreased (FIG. 4G). Additionally, the results of qRT-PCR were consistent with those of WB assay (FIG. 4H). These results demonstrated that TLPs could effectively reprogram M1 macrophages.

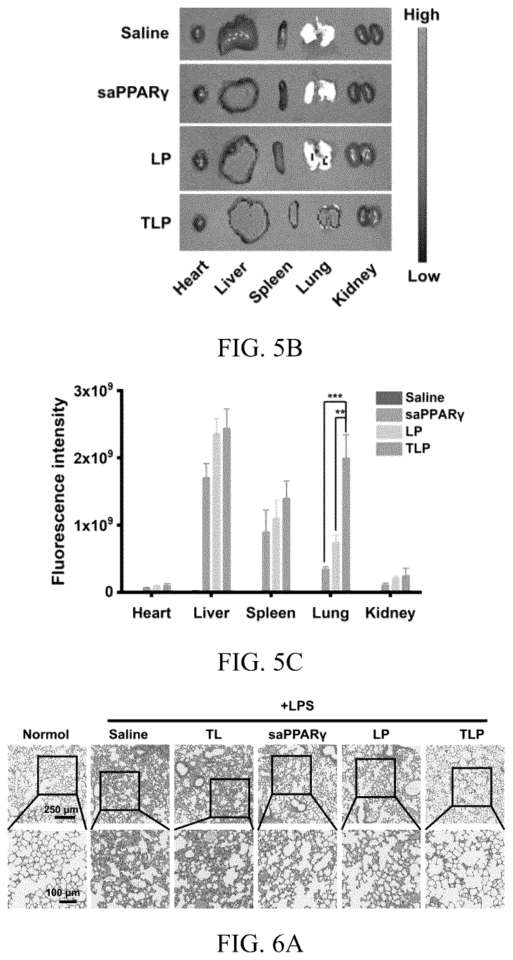

After TLP was administered to pneumonia mice via orbital injection, it was gradually accumulated in the lung tissues, and the fluorescence intensity in the lungs was progressively enhanced, with a strong fluorescence signal still detectable at 48 hours (FIG. 5A). After 48 hours, the mice were sacrificed, and the main visceral organs were harvested for re-imaging. Fluorescence was detected in the liver, spleen and lungs (FIG. 5B). The fluorescence intensity in the lungs of mice in the TLP group was the strongest among all groups (FIG. 5C). The above results indicate that TLP can be effectively targeted to the lungs and prolong the circulation time in vivo.

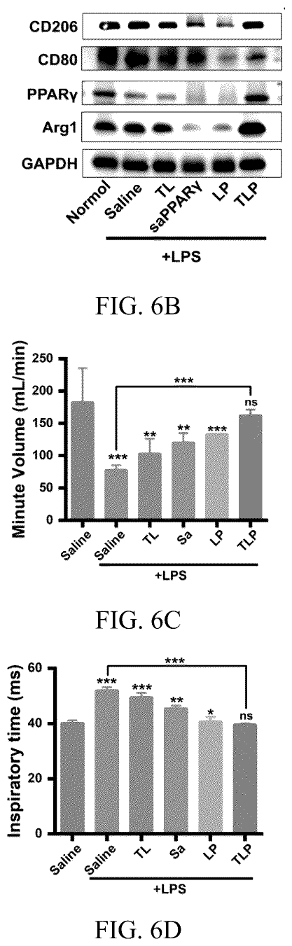

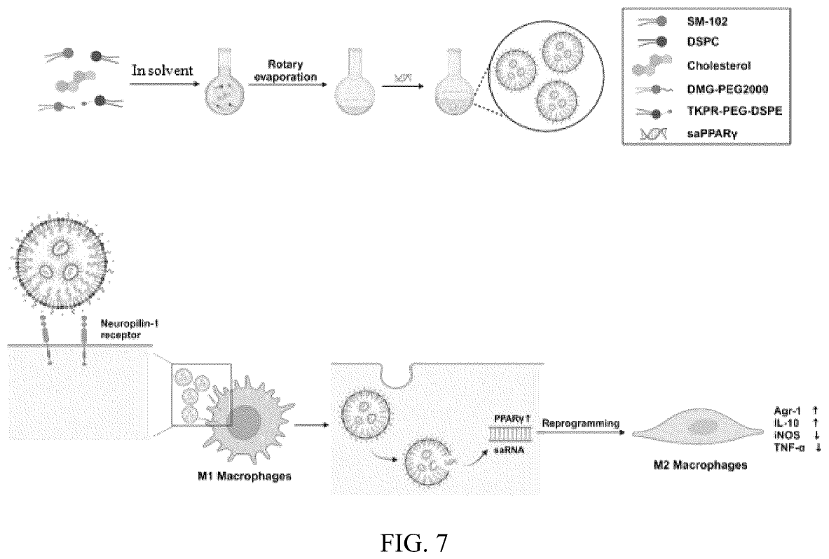

Pneumonia mice were administered via orbital injection every 3 days. Three days after the last administration, lung function parameters were detected, and the mice were sacrificed and dissected. Lung tissues were harvested, fixed, sectioned, and stained with H&E. The TL group showed almost no therapeutic effect, exhibiting a significant increase in immune cells and red blood cells in the alveoli and interstitial spaces, along with the formation of hyaline membranes and thickening of alveolar walls, which was consistent with the pathological changes observed in pneumonia mice treated with normal saline. The saRNA group and LP group showed weak therapeutic effects, whereas the TLP group presented thin alveolar walls and fewer inflammatory cells and red blood cells in the alveolar and interstitial regions, indicating that pneumonia was significantly alleviated (FIG. 6A). TLP also exhibited an improvement in lung function, which was evidenced by the MV and TI of pneumonia mice that were close to the levels of normal mice (FIGS. 6C and 6D). This suggested that TLP treatment significantly alleviated pneumonia and restored lung function. Tissue proteins were extracted for WB assay. The expression level of CD80, a marker of M1 macrophages, was significantly downregulated, while the expression levels of Arg1 and CD206, markers of M2-type macrophages, were significantly upregulated (FIG. 6B). This demonstrated that TLP could effectively reprogram macrophages in vivo.

The above-mentioned examples merely describe the preferred ways of the present disclosure and do not limit the scope of the present disclosure. Various modifications and improvements made to the technical solutions of the present disclosure by those skilled in the art without departing from the spirit of the present disclosure shall fall within the scope of protection determined by the claims of the present disclosure.

1. A preparation method of a macrophage-targeted small activating ribonucleic acid (saRNA) nanomedicine for inflammatory microenvironment, comprising the steps of:
(1) dissolving SM-102, 1,2-distearoyl-sn-glycero-3-phosphocholine (DSPC), cholesterol, 1,2-dimyristoyl-rac-glycero-3-methoxypolyethylene glycol-2000 (DMG-PEG2000) and TKPR-polyethylene glycol 2000-distearoylphosphatidylethanolamine (TKPR PEG2000-DSPE) in an organic solvent at a molar ratio of 50:10:37:1.5:1.5; removing the organic solvent by rotary evaporation; and adding 1-3 mL of buffer solution for hydration to adjust a total concentration of SM-102, DSPC, Cholesterol, DMG-PEG2000 and TKPR-PEG2000-DSPE in a mixed solution to 0.4-0.6 mg/mL, and forming a liposome solution; and
(2) diluting 0.8-1.2 μg saRNA in 0.8-1.2 μL of ribonuclease (RNase)-free water, and complexing the diluted saRNA with the liposome solution at a mass ratio of liposome solution to saRNA of 38-42:0.8-1.2, followed by standing at room temperature and centrifuging to obtain a precipitate, which is a macrophage-targeted saRNA nanomedicine for inflammatory microenvironment, wherein
sequences of sense and antisense strands of the saRNA are shown in SEQ ID NOs.3 and 4;
SEQ ID NO. 3: sense:
5′-CCAAUAGUCUAACUUAAAAdTdT-3′;
SEQ ID NO.4: anti-sense: 5′-UUUUAAGUUAGACUAUUGGdTdT-3 wherein
the organic solvent in step (1) is a mixed liquid of chloroform and methanol;
a volume ratio of chloroform and methanol in step (1) is 3:1;
the buffer solution in step (1) is 8-12 nM citrate buffer solution;
a total concentration of SM-102, DSPC, cholesterol, DMG-PEG2000, TKPR-PEG2000-DSPE in the mixed solution in step (1) is 0.5 mg/mL;
in step (2), 1 μg of saRNA is diluted in 1 μL of RNase-free water; and,
in step (2), a mass ratio of liposome solution to saRNA is 40:1.

2. A macrophage-targeted saRNA nanomedicine for inflammatory microenvironment prepared by the preparation method according to claim 1.

3. An application of the macrophage-targeted saRNA nanomedicine for inflammatory microenvironment according to claim 2 in preparing anti-pneumonia drugs.
